(19) United States
(12) Patent Application Publication
(10) Pub. No.: US 2022/0218062 A1
(43) Pub. Date: Jul. 14, 2022

(54) METHOD AND APPARATUS FOR REDUCING FACIAL EXPOSURE TO AIRBORNE GERMS

(71) Applicants: Troy PERRY, Victoria (CA); Jeff SHAW, Victoria (CA)

(72) Inventors: Troy PERRY, Victoria (CA); Jeff SHAW, Victoria (CA)

(21) Appl. No.: 17/375,594

(22) Filed: Jul. 14, 2021

Related U.S. Application Data
(63) Continuation-in-part of application No. 17/145,618, filed on Jan. 11, 2021, now abandoned.

(51) Int. Cl. A42B 1/017; A42B 1/244; A61L 9/20; A41D 13/11; A42B 3/04

(52) U.S. Cl. CPC A42B 1/017; A42B 1/244; A42B 3/0406; A41D 13/1184; A61L 9/20

(57) ABSTRACT

An apparatus for reducing facial exposure to airborne germs a UV-C light generator housed within a support housing and a UV-C output array mounted on headgear in an orientation to bathe a face of a person wearing the headgear with UV-C light. A fibre optic cable connects the UV-C generator with the UV-C output array, such that UV-C light generated by the UV-C light generator passes through the fibre optic cable to the UV-C output array.

### CROSS REFERENCE TO RELATED APPLICATIONS

This application is a Continuation-In-Part of U.S. patent application Ser. No. 17/145,618 filed Jan. 11, 2021, the entirety of which is incorporated by reference herein.

### FIELD

The present invention relates to a method for reducing facial exposure to airborne germs and an apparatus that has been developed in accordance with the teachings of the method.

### BACKGROUND

As the global population continues to grow and communities become increasingly urbanized, the global population continues to congregate to create denser living environments. With denser communities and increasing interactions between individuals comes the risk of increasing rates of human-transmissible infections and disease. In the past decade, outbreaks of virus and bacterial infections has caused the deaths of many. The SARS epidemic and the COVID-19 pandemic are examples among many. Whilst finding a treatment for these infections is important, finding ways to prevent infection is equally significant.

Currently, measures taken to prevent the spread of viral infections include frequent sanitation with chemical and physical agents, limiting physical interactions between individuals, erecting physical barriers to prevent spread of airborne particulates, and use of personal protective equipment. Personal Protective Equipment (PPE) includes masks, gloves, gowns, head gear with face shields and similar protective gear.

U.S. Pat. No. 6,843,964 (Yeh) titled: “Air Purifying Cap”, discloses a cap that generates anions to purify the air around a user's face.

### SUMMARY

According to one aspect, there is provided a method for reducing facial exposure to airborne germs. The method involves affixing at least one UV-C light generator to a person, and focusing the at least one UV-C light generator to bathe a face of the person with ultra-violet C light, thereby reducing facial exposure to airborne germs approaching eyes, nose and mouth of the person.

According to another aspect, there is provided an apparatus for reducing facial exposure to airborne germs. The apparatus includes a support and a mounting for mounting the support to a human body in a substantially horizontal orientation. At least one UV-C light generator is supported by the support. The at least one UV-C light generator is oriented to direct UV-C light in a substantially vertical orientation.

It is envisaged that more than one UV-C light generator will be used to create a light curtain. That will extend down the face from above, extend up the face from below or extend across the face from one of the sides. It is envisaged that the UV-C light generators will operate continuously, but there may be instances where intermittent operation would provide adequate protection.

In order to facilitate the desired positioning, it is envisaged that the support for the at least one UV-C light generator will be mounted to a head or shoulders of a person. The mounting could be some form of headgear that rests on the head of the person. It will be appreciated that there are numerous configurations of headgear that could be used, such as: a hat, a cap, a helmet, a face shield, a visor, to name just a few. The mounting could also be a form of collar that rests on the shoulders of the person, with the UV-C light generators projecting a light curtain vertically upward. It will also be appreciated that the UV-C light generators could project the light curtain across the face from left to right or from right to left. There will hereinafter be illustrated and described of each of these embodiments.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a perspective view of a first embodiment of apparatus for reducing facial exposure to airborne germs in accordance with the teachings of the method.

FIG. 2 is an exploded perspective view of the first embodiment illustrated in FIG. 1.

FIG. 3 is a perspective view of a second embodiment of apparatus for reducing facial exposure to airborne germs in accordance with the teachings of the method.

FIG. 4 is a perspective view of a third embodiment of apparatus for reducing facial exposure to airborne germs in accordance with the teachings of the method.

FIG. 5 is a perspective view of a fourth embodiment of apparatus for reducing facial exposure to airborne germs, considered a commercial embodiment, which combines various features of the first, second and third embodiments.

FIG. 6 is a side elevation view of the fourth embodiment illustrated in FIG. 5.

FIG. 7 is a side elevation view, in section, of a UV light generator from the fourth embodiment illustrated in FIG. 6.

### DETAILED DESCRIPTION

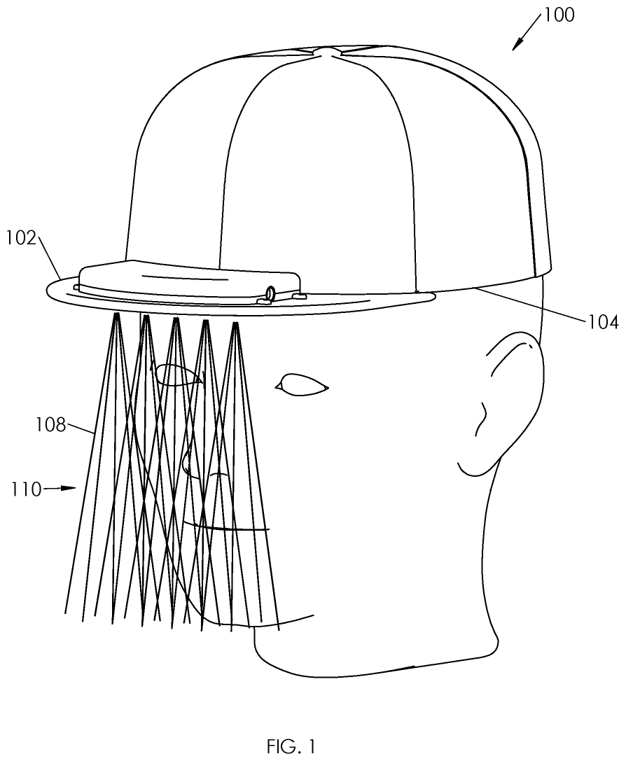

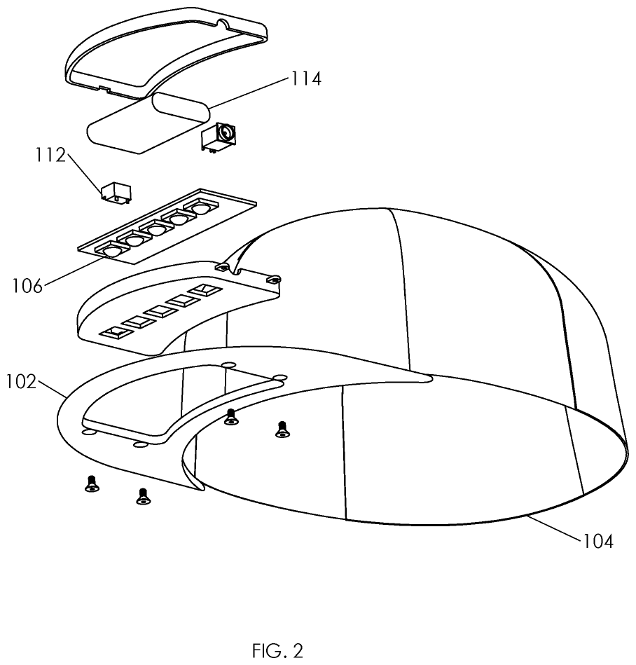

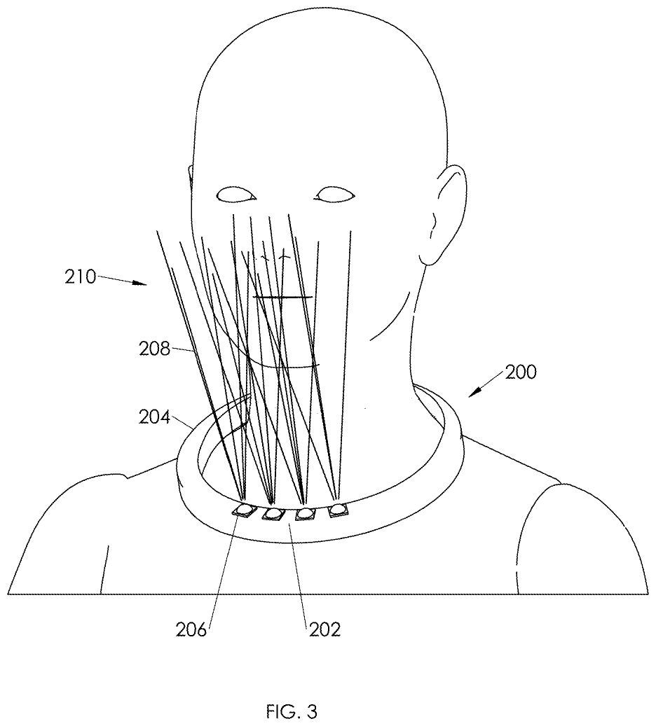

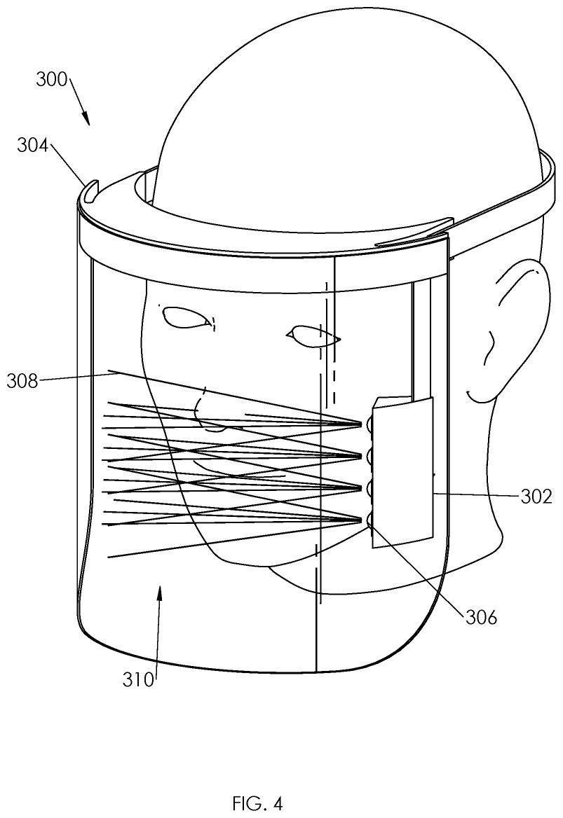

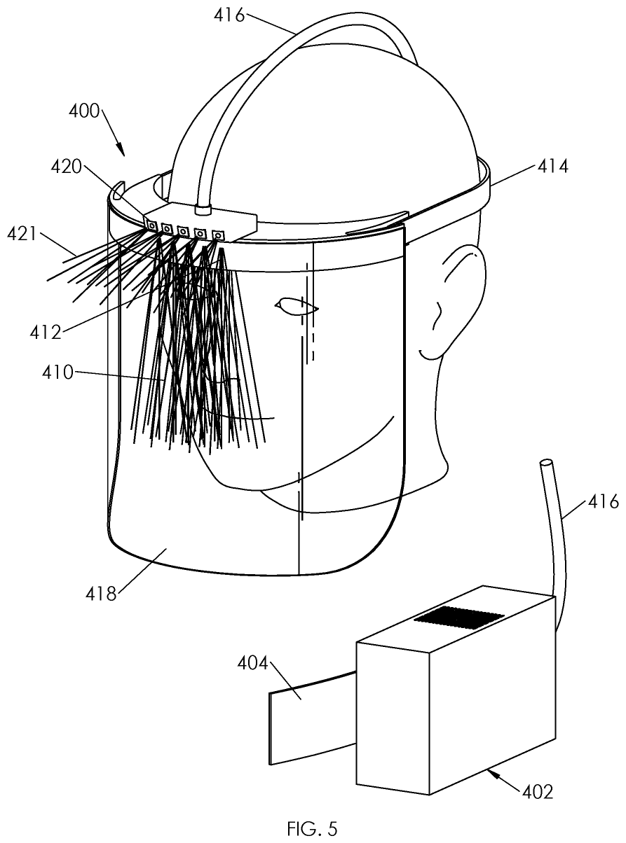

A first embodiment of apparatus for reducing facial exposure to airborne germs generally identified by reference numeral 100, will now be described with reference to FIG. 1 and FIG. 2. A second embodiment of apparatus for reducing facial exposure to airborne germs generally identified by reference numeral 200, will hereinafter be described with reference to FIG. 3. A third embodiment of apparatus for reducing facial exposure to airborne germs generally identified by reference numeral 300, will hereinafter be described with reference to FIG. 4. A fourth embodiment of apparatus for reducing facial exposure to airborne germs generally identified by reference numeral 400, will hereinafter be described with reference to FIG. 5 through FIG. 7.

First embodiment 100, second embodiment 200 and third embodiment 300 are examples of apparatus that have been developed to implement the teachings of the method. In broad terms that method involves affixing one or more UV-C light generator to a person, and focusing the one or more UV-C light generator to continuously bathe a face of the person with ultra-violet C light in a range of 100-280 nm. When the face is bathed in UV-C light, airborne germs approaching eyes, nose and mouth of the person are to a large extent neutralized.

It is envisaged that first embodiment 100 will be worn by a sports fan when attending sporting events. It is envisaged that second embodiment 200 will be worn by an entertainer when performing at a music concert or by a person attending a music concert where the wearing of hats would not be welcome. There are various environments in which public figures wish to remain safe, but wearing personal protective equipment is not practical.

This includes theatrical actors on stage and politicians at political rallies. It is envisaged that third embodiment 300 will provide an extra measure of protection for health professionals.

### Structure and Relationship of Parts of First Embodiment 100:

Referring to FIG. 1, first embodiment 100 includes a support 102, a mounting 104 for mounting support 102 to a human body in a substantially horizontal orientation, and at least one UV-C light generator 106 supported by support 102. The at least one UV-C light generator 106 is oriented to continuously bathe a face of the person with ultra-violet C light 108. Ultra-violet C light 108 is in the range of 100-280 nm. Universities have been experimenting with Ultra-violet C light in the range of 200-280 nm. For this reason, 200-280 nm is the preferred range.

As illustrated in FIG. 1, in first embodiment 100, there are five UV-C light generators 106 creating a light curtain 110. However, it will be appreciated that there could be any number of UV-C light generators 106 used. The five UV-C light generators 106 are secured to the head of the person. Mounting 104 is a headgear and support 102 is a visor that projects substantially horizontally from the headgear. It will be appreciated that there are numerous configurations of headgear that could be used, such as: a hat, a cap, a helmet, etc. First embodiment 100 also includes a switch 112 that switches the five UV-C light generators 106 on and off.

As illustrated in FIG. 2, first embodiment 100 also includes a battery pack 114 that supplies power to the five UV-C light generators 106.

### Operation of First Embodiment 100:

Referring to FIG. 1, a user starts by placing first embodiment 100 on their head, such that support 102 is oriented towards the front of the user's face. The user then turns on the five UV-C light generators 106 using switch 112. The user then makes positional adjustments as needed, such that the five UV-C light generators 106 are creating light curtain 110 directly in front of and slightly on to the user's face. The user may then proceed to go about their usual daily activities. Over time, light curtain 110 created by the five UV-C light generators 106 will neutralize many of the harmful germs that would otherwise come in contact with the user's face.

It is envisaged that first embodiment 100 will be worn by a sports fan when attending sporting events. The casual nature and design of first embodiment 100 (being headgear in a cap or hat form) enables a user to wear first embodiment 100 to many casual outings and events, as well as when a user is out and about in their usual daily activities.

### Structure and Relationship of Parts of Second Embodiment 200:

Referring to FIG. 3, second embodiment 200 includes a support 202, a mounting 204 for mounting support 202 to a human body in a substantially horizontal orientation, and at least one UV-C light generator 206 supported by support 202. The at least one UV-C light generator 206 is oriented to continuously bathe a face of the person with ultra-violet C light 208. Ultra-violet C light 208 is in the range of 100-280 nm. Universities have been experimenting with Ultra-violet C light in the range of 200-280 nm. For this reason, 200-280 nm is the preferred range.

As illustrated in FIG. 3, in second embodiment 200, there are four UV-C light generators 206 creating a light curtain 210. However, it will be appreciated that there could be any number of UV-C light generators 206 used. Mounting 204 is a collar and support 202 is supported by the collar. It will be appreciated that, for women, mounting could be in the form of a necklace.

### Operation of Second Embodiment 200:

Referring to FIG. 3, a user starts by placing second embodiment 200 around their neck, such that support 202 is oriented towards the front of the user's face. The user then turns on the four UV-C light generators 206 and makes positional adjustments as needed, such that the four UV-C light generators 206 are creating light curtain 210 directly in front of and slightly on to the user's face. The user may then proceed to go about their usual daily activities. Over time, light curtain 210, created by the four UV-C light generators 206, will neutralize many of the harmful germs that would otherwise come in contact with the user's face.

It is envisaged that second embodiment 200 will be worn by an entertainer when performing at a music concert or by a politician, where the audience has paid to view the personality. The nature and design of second embodiment 200 (being a device that is worn around the neck, such as a collar or necklace) enables a user to have unobstructed vision while still benefiting from the neutralizing effects of light curtain 210, with airborne germs approaching eyes, nose and mouth of the person being to a large extent neutralized by UV-C light. Second embodiment 200 would also be appropriate in and well-suited to situations where headgear is not appropriate or permitted (e.g., in some school settings or at more formal events).

### Structure and Relationship of Parts of Third Embodiment 300:

Referring to FIG. 4, third embodiment 300 includes a support 302, a mounting 304 for mounting support 302 to a human body, and at least one UV-C light generator 306 supported by support 302. The at least one UV-C light generator 306 is oriented to continuously bathe a face of the person with ultra-violet C light 308. Ultra-violet C light 308 is in the range of 200-280 nm.

As illustrated in FIG. 4, in third embodiment 300, there are four UV-C light generators 306 creating a light curtain 310. However, it will be appreciated that there could be any number of UV-C light generators 306 used. Mounting 304 is a headgear and support 302 is a face shield that extends substantially vertically from the headgear.

### Operation of Third Embodiment 300:

Referring to FIG. 4, a user starts by placing third embodiment 300 on their head, such that support 302 is oriented towards the front of the user's face. The user then turns on the four UV-C light generators 306 and makes positional adjustments as needed, such that the four UV-C light generators 306 are creating light curtain 310 directly in front of and slightly on to the user's face. The user may then proceed to go about their usual daily activities. Over time, light curtain 310 created by the five UV-C light generators 306 will neutralize many of the harmful germs that would otherwise come in contact with the user's face.

It is envisaged that third embodiment 300 will be worn by health professionals in settings where an extra measure of protection is desirable to supplement the use of a face shield.

### Structure and Relationship of Parts of Fourth Embodiment 400 (Commercial Version):

In the course of working on a commercial version of the apparatus for reducing facial exposure to airborne germs, all three embodiments were critically reviewed. It will be apparent that the commercial version that will hereafter be described is a composite of the best features selected from the earlier embodiments. There are, however, two new aspects.

One aspect that was considered was the positioning of the UV-C generator. When secured directly to the headgear, the UV-C generator added weight which made the headgear cumbersome. There was also a concern expressed on possible adverse health effects of long term use of the apparatus with the UV-C generator positioned against the head or throat. It was then realized that while a UV-C output array had to be mounted on headgear to bathe a face of a person wearing the headgear with UV-C light, the UV-C generator itself could be positioned elsewhere on the human body with a fibre optic cable connecting the UV-C generator with the UV-C output array, as will be hereinafter described.

Another aspect incorporated into the commercial embodiment was a secondary UV-C output array. It was felt that it would be advantageous to contemporaneously sterilize a focal region in front of the headgear. Assuming that the person wearing the apparatus is a health care professional, it is envisaged that a wound of a patient may be positioned in the focal region.

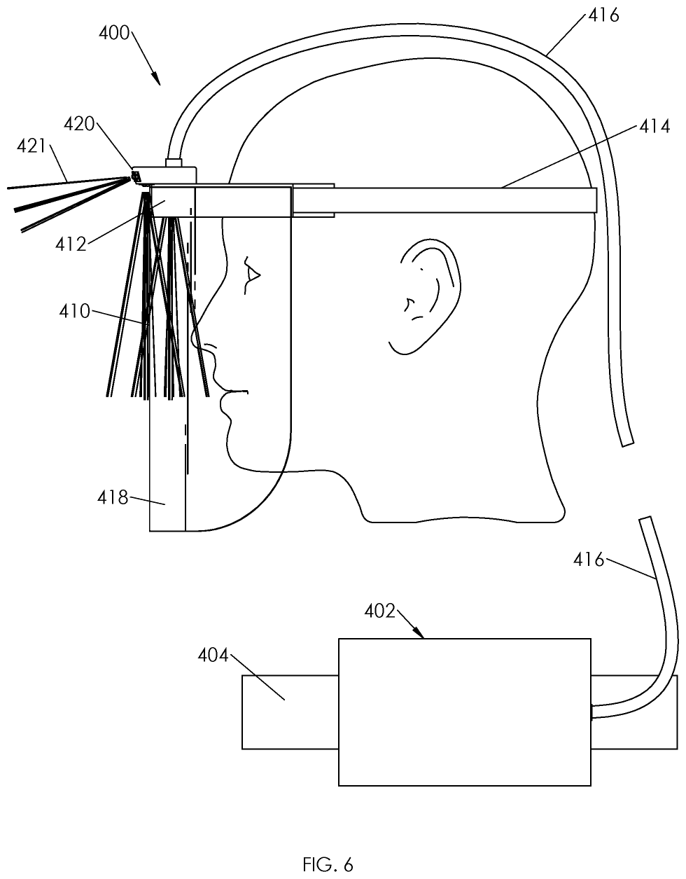

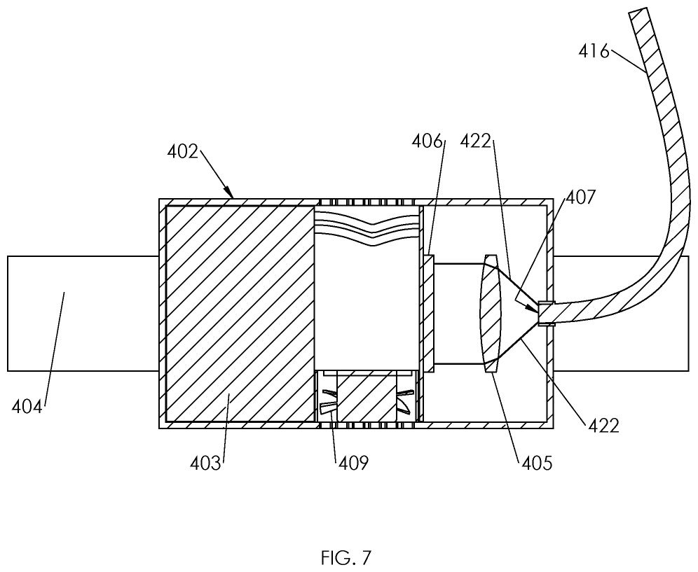

Referring to FIG. 5 and FIG. 6, fourth embodiment 400 has a support housing 402. Referring to FIG. 7, a UV-C light generator 406 is housed within support housing 402, along with a battery 403 to provide power to UV-C light generator 406 and a focusing lens 405 to focus beams of UV-C light 422 generated by UV-C light generator 406 at a light output aperture 407. Referring to FIG. 5 and FIG. 6, a mounting, in the form of a belt 404 is provided for mounting support housing 402 to a human body. It is envisaged that belt 404 is a waist belt that will be placed around a human waist, but belt 404 could also be attached around a human limb, such as a leg or an arm. In order to avoid heat build up, it is preferred that a fan 409 be provided to create and air flow through support housing 402 and thereby dissipate heat.

Referring to FIG. 5 and FIG. 6, there is provided a primary UV-C output array 412 mounted on headgear 414. Primary UV-C output array 412 is oriented to create a light curtain 410 which bathes a face of a person wearing headgear 414 with UV-C light. After experimenting with various ways of orienting light curtain 410, it is preferred that primary UV-C output array 412 is oriented to create a light curtain 410 will projects vertically downwardly from headgear 414.

Referring to FIG. 5 and FIG. 5, a fibre optic cable 416 connects support housing 402 with primary UV-C output array 412, such that UV-C light exiting light output aperture 407 passes through fibre optic cable 416 to primary UV-C output array 412.

Referring to FIG. 5 and FIG. 6, after experimenting with various types of configurations intended for medical use, it is preferred that headgear 414 has a transparent face shield 418 that extends downwardly substantially vertically from 414 headgear. Face shield 418 provides a barrier between the user (for example a medical worker) and others (for example a patient).

Referring to FIG. 5 and FIG. 6, when used by medical workers it is preferred that there be provided a secondary UV-C output array 420. Secondary UV-C output array 420 is mounted on headgear 414 and is oriented outwardly to create a secondary light curtain 421 to bathe in ultra-violet C light a focal region in front of and spaced from headgear 414. When secondary UV-C output array 421 is included, fibre optic cable 416 connects support housing 402 with both primary UV-C output array 412 and secondary UV-C output array 420, such that UV-C light exiting light output aperture 407 passes through fibre optic cable 416 to both primary UV-C output array 412 and secondary UV-C output array 420.

### Operation of Fourth Embodiment 400:

Referring to FIG. 5 and FIG. 6, a user starts by mounting support housing 402 to his or her body by securing belt 404 around his or her waist. Headgear 414 is placed on his or her head, with transparent face shield 418 extending vertically downward. Referring to FIG. 7, the user then turns on UV-C light generator 406. Beams of light 422 generated by UV-C light generator 406 are focused by focusing lens 405 at light output aperture 407. UV-C light exiting light output aperture 407 passes through fibre optic cable 416 to primary UV-C output array 412 and secondary UV-C output array 420. Primary UV-C output array 412 is oriented to create light curtain 410 that projects vertically downwardly from headgear 414 roughly parallel to transparent face shield 418. Secondary UV-C output array 420 is oriented outwardly to create secondary light curtain 421 that projects into a focal region in front of and spaced from headgear 414. Light curtain 410 neutralizes many of the harmful germs that would otherwise come in contact with the user's face. Light curtain 421 serves to neutralize many harmful germs in the focal region.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

The scope of the claims should not be limited by the illustrated embodiments set forth as examples, but should be given the broadest interpretation consistent with a purposive construction of the claims in view of the description as a whole.

What is claimed is:

1. An apparatus for reducing facial exposure to airborne germs, comprising:
a support housing;
a UV-C light generator housed within the support housing, along with a battery to provide power to the UV-C light generator and a focusing lens to focus UV-C light generated by the UV-C light generator at a light output aperture;
a mounting for mounting the support housing to a human body;
a primary UV-C output array mounted on headgear, the primary UV-C output array being oriented to bathe a face of a person wearing the headgear with UV-C light; and
a fibre optic cable connecting the support housing with the primary UV-C output array, such that UV-C light exiting the light output aperture passes through the fibre optic cable to the primary UV-C output array.

2. The apparatus of claim 1, wherein the mounting for the support housing is a waist belt.

3. The apparatus of claim 1, wherein the primary UV-C output array is oriented vertically downwardly from the headgear.

4. The apparatus of claim 1, wherein the headgear has a face shield that extends substantially vertically from the headgear.

5. The apparatus of claim 1, wherein
a secondary UV-C output array is mounted on the headgear, the secondary UV-C output array being oriented outwardly to bathe in ultra-violet C light a focal region in front of and spaced from the headgear; and
the fibre optic cable connects the support housing with both the primary UV-C output array and the secondary UV-C output array, such that UV-C light exiting the light output aperture passes through the fibre optic cable to both the primary UV-C output array and the secondary UV-C output array.

6. An apparatus for reducing facial exposure to airborne germs, comprising:
a support housing,
a UV-C light generator housed within the support housing;
a UV-C output array mounted on headgear and oriented to bathe a face of a person wearing the headgear with UV-C light; and
a fibre optic cable connecting the UV-C light generator with the UV-C output array, such that UV-C light generated by the UV-C light generator passes through the fibre optic cable to the UV-C output array.
